(19) United States
(12) Patent Application Publication
(10) Pub. No.: US 2011/0082709 A1
(43) Pub. Date: Apr. 7, 2011

(54) SYSTEM AND DEVICE AND METHOD FOR BLOOD SUGAR LEVEL ANALYSIS AND COMPUTER READABLE RECORDING MEDIUM STORING COMPUTER PROGRAM PERFORMING THE METHOD

(75) Inventors: Huan-Chung LI, MIAOLI COUNTY (TW); Shun-Chi CHUNG, TAIPEI COUNTY (TW)

(73) Assignee: INSTITUTE FOR INFORMATION INDUSTRY, TAIPEI (TW)

(21) Appl. No.: 12/628,224

(22) Filed: Dec. 1, 2009

(30) Foreign Application Priority Data
Oct. 2, 2009 (TW) 98133597

(51) Int. Cl. G06Q 50/00; A61B 5/145; G06F 17/30; G06F 7/10

(52) U.S. Cl. 705/3; 600/365; 705/2; 707/752; 707/769; 707/E17.044; 707/E17.014; 707/802

(57) ABSTRACT

A blood sugar level analysis system includes a database, an input device, a processing device and a display device. The processing device is connected to the database, the input device and the display device. The processing device includes a blood sugar recording module and an evaluation module. The blood-sugar recording module obtains at least one measured blood sugar level of a user taken within at least one measurement time period of a day, and records the measured blood sugar level into the database. The evaluation module obtain the measured blood sugar level taken within the measurement time period for each day in an evaluation time span from the database, and bases on the measured blood sugar level(s) obtained to evaluate a abnormal blood sugar level risk of the user within the measurement time period, and the risk is provided for display at the display device.

### RELATED APPLICATIONS

This application claims priority to Taiwan Application Serial Number 98133597, filed Oct. 2, 2009, which is herein incorporated by reference.

### BACKGROUND

1. Field of Invention

The present invention relates to a system, a device and a method for blood sugar level analysis. More particularly, the present invention relates to a blood sugar level analysis system, a blood sugar level analysis device and a blood sugar level analysis method for evaluating abnormal blood sugar level risks occurring at various time periods.

2. Description of Related Art

Due to rich economy and social pattern transformation, the life styles and dietary habits of people have been changed, resulting in that diabetes becomes one of ten major causes of domestic fatalities. Diabetes is a chronic metabolic disease associated with genetics. It is commonly considered that the diabetes is caused by the cross influences of genetics, environments, chemical toxins and microorganisms. The pathogenesis of the diabetes includes insufficient insulin secreted by the human pancreas or insulin resistance in human body, which lowers or even disabling the capability of utilizing carbohydrate, thus resulting in the symptoms of high blood sugar level and sugar in urine, also causing abnormal fat and protein metabolism.

For diabetes patients, active diet control and nutrition management are the most critical ways to control their blood sugar levels. Hence, regardless of light or heavy diabetes symptoms, it is a must to use the diet control collaborating with proper exercise to assist the medical treatment for effectively controlling blood sugar levels. The diets for diabetes patients are based on well-balanced dietaries, and then the servings of six classifications of food and the proportionalities of three major nutritional components such as carbohydrates, proteins and fats are adjusted in accordance with each individual's needs, so as to achieve the dietary principle for controlling blood sugar levels.

For tracing and controlling the blood sugar levels of diabetes patients, a conventional skill enables the diabetes patients to consistently measure and record their blood sugar levels by themselves so as to achieve self cares. However, such conventional skill merely can let the diabetes patients know their blood sugar high/low levels, but cannot let them be aware of whether they have a high risk of abnormal blood sugar level; the factors causing the fluctuations of their blood sugar levels; and the efficacies of their self cares. Further, the diabetes patients usually seek the assistance of nutritionists to design diets by evaluating their individual physical conditions and illness situations, and also to teach them how to have balanced diets for controlling their blood sugar levels. However, such methods cannot provide personal diet suggestions based on personal favorites.

### SUMMARY

Hence, the present invention provides a blood sugar level analysis system and its applications for using measured blood sugar levels of a user taken within specific measurement time periods to analyze abnormal blood sugar level risks of the user within the specific measurement time periods.

Further, the system and its applications of the present invention can be used to analyze the abnormal blood sugar level risks of in accordance with the measured blood sugar levels.

Further, the system and its applications of the present invention can be used to record the user's diet habits, and provide personal diet suggestions in accordance with the user's abnormal blood sugar level risks and diet habits.

In one embodiment, a blood sugar level analysis system includes a database, an input device, a processing device and a display device. The processing device is connected to the database, the input device and the display device. The processing device includes a blood sugar recording module and an evaluation module. The blood sugar recording module may obtain at least one measured blood sugar level of a user taken within at least one measurement time period of a day, and record the measured blood sugar level into the database. The evaluation module may obtain the measured blood sugar levels taken within the measurement time period for each day in an evaluation time span from the database, and evaluate at least one abnormal blood sugar level risk of the user within the at least one measurement time period respectively in accordance with the measured blood sugar level(s) obtained, and the abnormal blood sugar level risk of the user is provided for display at the display device.

In another embodiment, a blood sugar level analysis device includes a blood sugar testing element, a storage element, an input element, a processing element and a display element. The processing element is connected to the blood sugar testing element, the storage element and the input element. The processing element includes a blood sugar recording module and an evaluation module. The blood sugar testing element is used to measure a blood sugar level of a user. The blood sugar recording module may obtain via the blood sugar testing element at least one measured blood sugar level of the user taken within at least one measurement time period of a day, and recording the measured blood sugar level into the storage element. The evaluation module may obtain from the storage element the at least one measured blood sugar level taken within the at least one measurement time period for each day in an evaluation time span, and evaluate at least one abnormal blood sugar level risk of the user within the at least one measurement time period respectively in accordance with the at least one measured blood sugar level obtained, and the abnormal blood sugar level risk of the user is provided for display at the display element.

In another embodiment, a blood sugar level analysis method includes the steps of: testing and recording into a database at least one measured blood sugar level of a user taken within at least one measurement time period of a day; obtaining the at least one measured blood sugar level taken within the at least one measurement time period for each day in an evaluation time span from the database; and evaluating at least one abnormal blood sugar level risk of the user within the at least one measurement time period in accordance with the at least one measured blood sugar level obtained.

In another embodiment, a computer-readable recording medium storing a computer program performing a blood sugar level analysis method. The blood sugar level analysis method includes the steps of: testing and recording into a database at least one measured blood sugar level of a user taken within at least one measurement time period of a day; obtaining the at least one measured blood sugar level taken within the at least one measurement time period for each day in an evaluation time span from the database; and evaluating at least one abnormal blood sugar level risk of the user within the at least one measurement time period respectively in accordance with the at least icy one measured blood sugar level obtained.

The applications of the embodiments of the present invention can advantageously allow a user to learn his/her abnormal blood sugar level risks within the respective measurement time periods; further remind the user of the specific measurement time periods within which his/her blood sugar control is poor and needs further attention; and provide a recommended diet list in accordance with the user's diet record and measured blood sugar levels, thereby satisfying the user's personal diet favorites or physical conditions.

It is to be understood that both the foregoing general description and the following detailed description are examples, and are intended to provide further explanation of the invention as claimed.

### BRIEF DESCRIPTION OF THE DRAWINGS

These and other features, aspects, and advantages of the present invention will become better understood with regard to the following description, appended claims, and accompanying drawings where:

FIG. 1 is a functional block diagram showing a blood sugar level analysis system according to an embodiment of the present invention;

FIG. 2 is a functional block diagram of the processing device 140 shown in FIG. 1;

FIG. 3A is a schematic diagram showing the appearance of a blood sugar level analysis device according to another embodiment of the present invention;

FIG. 3B is a functional block diagram showing a blood sugar level analysis device according to the embodiment of the present invention;

FIG. 3C is a functional block diagram of the processing element 340 shown in FIG. 3B; and

FIG. 4 is a flow chart showing a blood sugar level analysis method according to another embodiment of the present invention.

### DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

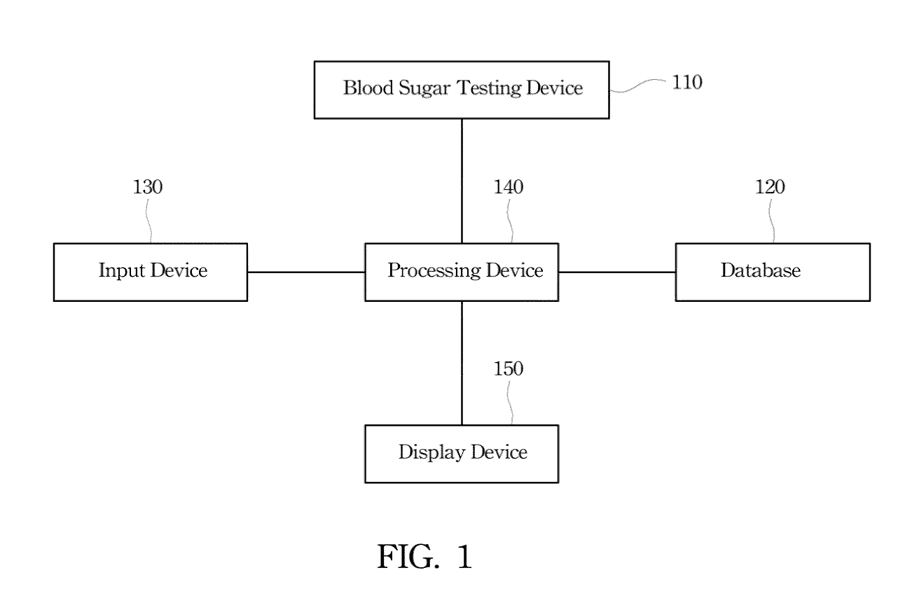

Referring to FIG. 1, FIG. 1 is a functional block diagram showing a blood sugar level analysis system according to an embodiment of the present invention. A blood sugar level analysis system 100 detects and records measured blood sugar levels of a user within specific measurement time periods respectively, and bases on the user's measured blood sugar levels recorded within an evaluation time span to analyze abnormal blood sugar level risks of the user within the specific measurement time periods.

The blood sugar level analysis system 100 includes a database 120, an input device 130, a processing device 140 and a display device 150. The processing device 140 is connected to the database 120, the input device 130 and the display device 150. The input device 130 can be a keyboard, a mouse, a touch screen, a data transmission interface or any other device with the function for receiving external input data. Further, the processing device 140 can be a computer or any other electronic device with the functions of processor.

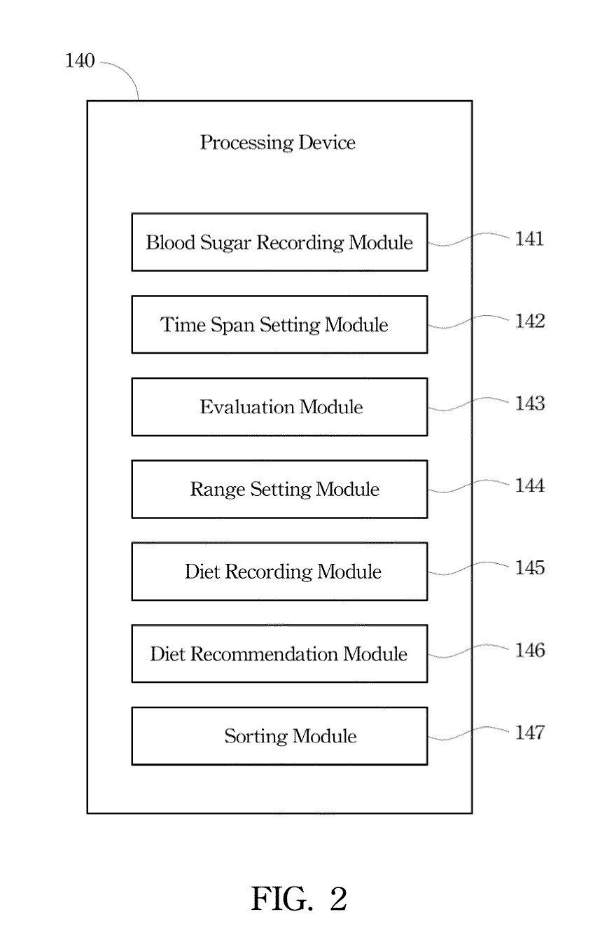

FIG. 2 is a functional block diagram of the processing device 140 shown in FIG. 1. Please refer to FIG. 1 and FIG. 2 simultaneously. The processing device 140 includes a blood sugar recording module 141 and an evaluation module 143. The blood sugar recording module 141 may obtain at least one measured blood sugar level of a user taken within at least one measurement time period of a day, and record the measured blood sugar level into the database 120, wherein the at least one measurement time period can be at least one meal periods such as before and/or after breakfast, before and/or after lunch, or before and/or after supper. Besides, the user also may schedule other time periods of a day as the measurement time periods for recoding the measured blood sugar levels.

The blood sugar level analysis system 100 may further include a blood sugar testing device 110 connected to the processing device 140, wherein the blood sugar testing device 110 can be used to measure at least one measured blood sugar level of the user within the at least one time periods, and the at least one measured blood sugar level are available to the blood sugar recording module 141. The blood sugar testing device 110 can be a commercial blood sugar meter, a palm-held or hand-held blood sugar meter or any other type of blood sugar testing device. The blood sugar testing device 110 can be connected to the blood sugar level analysis system 100 wiredly or wirelessly for data transmission. Besides, the blood sugar testing device 110 can be a blood sugar testing element which is built in the blood sugar level analysis system 100 and electrically connected to the process device 140. The user also may input his/her measured blood sugar levels by himself/herself via the input device 130, and the measured blood sugar levels inputted are available to the blood sugar recording module 141.

Further, the processing device 140 may include a time span setting module 142 for obtaining the evaluation time span from the input device 130. The user may use the input device 130 to select several dates from the dates listed in the database 120 on which the measured blood sugar levels have been recorded, and use the dates selected as the evaluation time span. Then, the evaluation module 143 obtains the measured blood sugar level(s) taken within the measurement time period(s) for each day in the evaluation time span from the database 120.

Thereafter, the evaluation module 143 evaluates at least one abnormal blood sugar level risk of the user within the at least one measurement time period respectively in accordance with the measured blood sugar level(s) obtained, and the abnormal blood sugar level risk of the user is provided for display at the display device 150. In some embodiments, the processing device 140 and the display device 150 can be two electrically-connected devices disposed in one electronic apparatus. In other embodiments, the processing device 140 also can be linked to the display device 150 at a remote user site via the Internet. For example, the user can use a cell phone, a personal digital assistant (PDA) or any other display device with a networking function as the display device 150, and link the display device to the processing device 140 via the Internet.

Further, the processing device 140 may include a range setting module 144 used for obtaining a target blood sugar level range from the input device 130. For example, a user, a medical care person or a nutritionist may enter his/her personal target blood sugar level range via the input device 130, wherein the target blood sugar level range can be the data provided by the medical care person in accordance with the user's physical conditions. Alternatively, a target blood sugar level range suggested by health organizations or medical care organizations for common people can be stored in the database 120 in advance and predetermined as the target blood sugar level range.

The greater the difference between the user's measured blood sugar level and the target blood sugar level range is, the higher the abnormal blood sugar level risk of the user is, and vice versa, the smaller the difference between the user's measured blood sugar level and the target blood sugar level range is, the lower the abnormal blood sugar level risk of the user is. Therefore, the evaluation module 143 may evaluate the abnormal blood sugar level risk of the user by computing the difference between the user's measured blood sugar level and the target blood sugar level range.

In another embodiment of risk evaluation, when a meal period is used as the measurement time period, the blood sugar recording module 141 may obtain and record a before-meal blood sugar level and an after-meal blood sugar level of the user respectively, wherein the greater the difference between the before-meal blood sugar level and the after-meal blood sugar level is, the higher the abnormal blood sugar level risk of the user is, and vice versa, the smaller the difference between the before-meal blood sugar level and the after-meal blood sugar level is, the lower the abnormal blood sugar level risk of the user is. Therefore, the evaluation module 143 may evaluate the abnormal blood sugar level risk of the user in accordance with the difference between the before-meal blood sugar level and the after-meal blood sugar level. For example, a difference between a blood sugar level before a certain meal of a day and a blood sugar level after the certain meal is computed, and then the abnormal blood sugar level risk is determined by checking if the difference is beyond a specific range. Alternatively, an average value of differences between before-meal blood sugar levels and after-meal blood sugar levels is used as a standard value, and then a difference of the blood sugar levels before and after a certain meal of a specific date is compared with the standard value, thereby evaluating the abnormal blood sugar level risk. In other embodiments, the evaluation module 143 may apply other methods of evaluating abnormal blood sugar level risks to evaluate the user's abnormal blood sugar level risk within the measurement time period, and thus the embodiments of the present invention are not limited to the methods described above.

When the user sets up a plurality of measurement time periods, the processing device 140 may further include a sorting module 147 for sorting the abnormal blood sugar level risks of the user within the respective measurement time periods so as to generate a sorted risk list provided for display at the display device 150. Thus, through the sorted risk list, the user can understand within which measurement time periods he/she has higher abnormal blood sugar level risks. Subsequently, the user can manage his/her blood sugar levels with respect to the measurement time periods within which he/she has higher abnormal blood sugar level risks.

Further, the processing device 140 may further include a diet recording module 145 and a diet recommendation module 146. The diet recording icy module 145 may record diet records of the user made within the respective measurement time periods into the database 120, wherein the diet records may include a food list, a medicine list or other diet-related records showing what the user had eaten within the respective measurement time periods. The diet recommendation module 146 may select at least one date on which the at least one measured blood sugar level obtained for the at least one measurement time period falls within the target blood sugar level range as at least one target met date. Then, the diet recommendation module 146 may obtain from the database 120 the at least one diet record of the user made within the at least one measurement time period of the at least one target met date, thereby generating at least one recommended diet list in accordance with the at least one diet record obtained. In other words, the diet records of the user meeting the target blood sugar level range can be used as a diet reference for the user to follow in the future. Besides, since the recommended diet list is generated in accordance with the user's diet records, it can be better fit to the user's diet favorites or physical conditions.

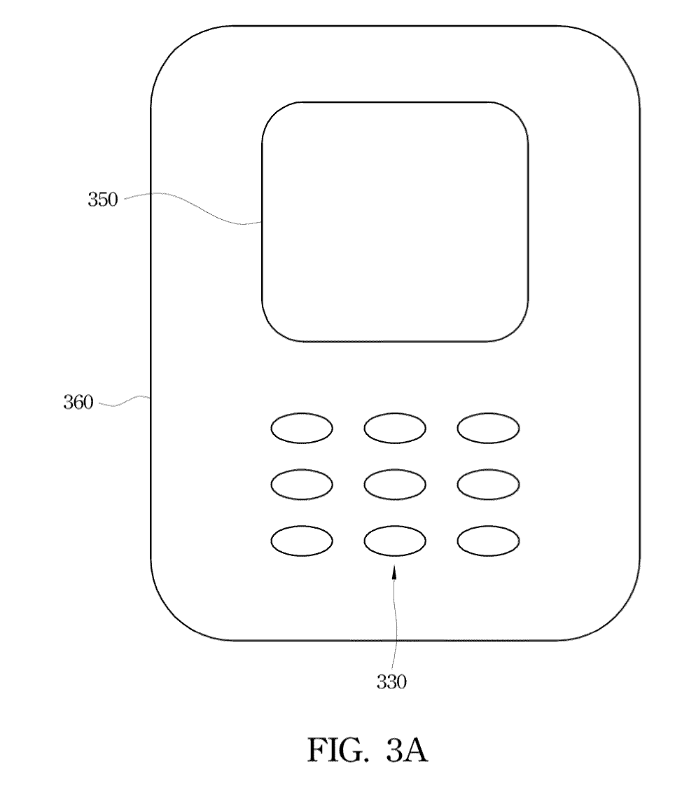

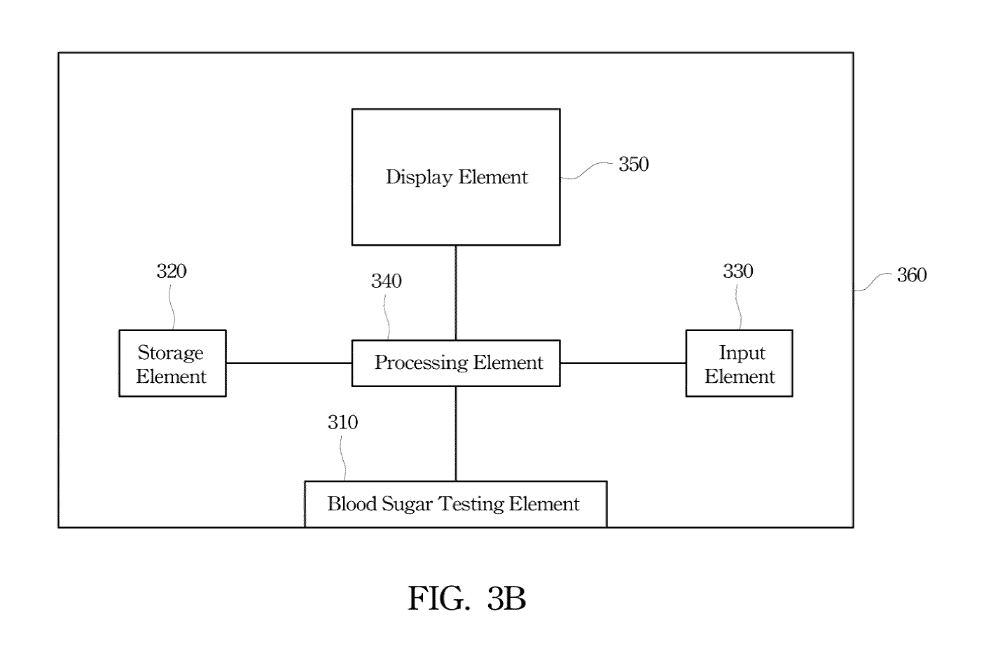

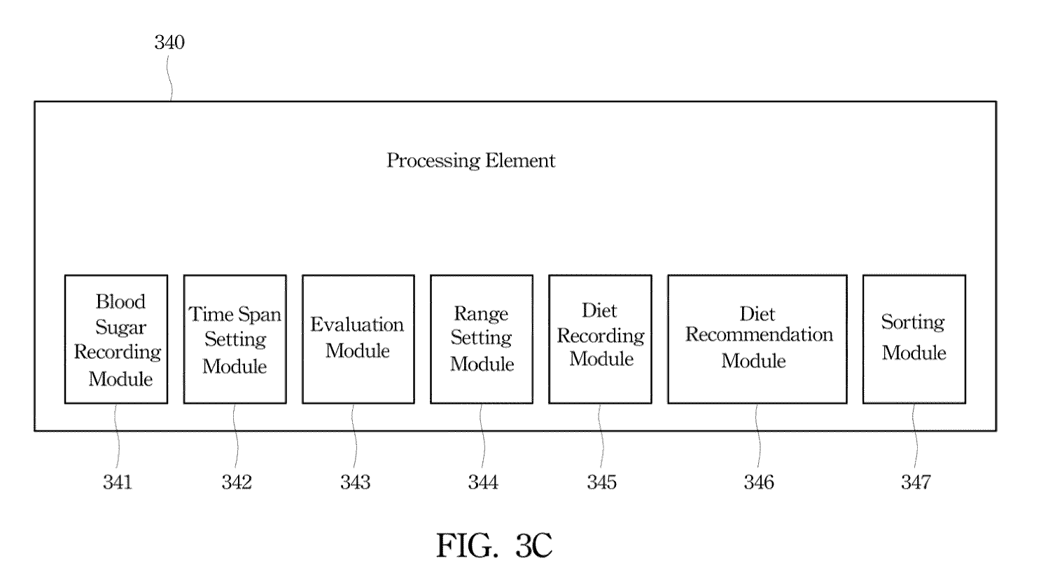

Referring to FIG. 3A and FIG. 3B, FIG. 3A and FIG. 3B are schematic diagram and a functional block diagram respectively showing the appearance and structure of a blood sugar level analysis device according to another embodiment of the present invention. A blood sugar level analysis device 300 detects and records measured blood sugar levels of a user within specific measurement time periods respectively, and bases on the user's measured blood sugar levels recorded within an evaluation time span to analyze abnormal blood sugar level risks of the user within the specific measurement time periods.

The blood sugar level analysis device 300 includes a blood sugar testing element 310, a storage element 320, an input element 330, a processing element 340 and a display element 350. The processing element 340 is connected to the storage element 320, the input element 330 and the display element 350. The storage element 320 can be a hard disk, a flash memory or any other type of storage element which can perform data accessing. In this embodiment, the input element 330 is a plurality of push buttons. However, in another embodiment, the input element 330 can be a keypad, a keyboard, a mouse, a touch screen, a voice input interface, a data transmission interface or any other element with the function for receiving external input data. Further, the blood sugar level analysis device 300 may include a housing 360 for installing the blood sugar testing element 310, the storage element 320, the input element 330, the processing element 340 and the display element 350, thereby allowing the user to carry the blood sugar level analysis device 300 conveniently and to measure blood sugar levels and perform analysis anytime.

FIG. 3C is an embodiment of the processing element 340 shown in FIG. 3B. Please refer to FIG. 3B and FIG. 3C simultaneously. The processing element 340 includes a blood sugar recording module 341 and an evaluation module 343. The blood sugar recording module 341 may obtain at least one measured blood sugar level of a user taken within at least one measurement time period of a day, and record the measured blood sugar level into the storage element 320, wherein the setup of the measurement time period can be referred to the above description applied to the blood sugar level analysis system 100.

Then, the evaluation module 343 obtains from the storage element 320 the measured blood sugar level(s) taken within the measurement time period(s) for each day in an evaluation time span. Thereafter, the evaluation module 343 evaluates at least one abnormal blood sugar level risk of the user within the at least one measurement time period respectively in accordance with the measured blood sugar level(s) obtained, and the abnormal blood sugar level risk of the user is provided for display at the display element 350. Further, the processing element 340 may include a time span setting module 342 for obtaining the evaluation time span from the input element 330. Accordingly, the user may use the input element 330 to select several dates from the dates stored in the storage element 320 on which the measured blood sugar levels have been recorded, and use the dates selected as the evaluation time span.

The processing element 340 may include a range setting module 314 used for obtaining a target blood sugar level range from the input element 330. In other words, the user may use the input element 330 to enter his/her personal target blood sugar level range, wherein the setup of the target blood sugar level range can be referred to the above description applied to the blood sugar level analysis system 100. The greater the difference between the user's measured blood sugar level and the target blood sugar level range is, the higher the abnormal blood sugar level risk of the user is, and vice versa, the smaller the difference between the user's measured blood sugar level and the target blood sugar level range is, the lower the abnormal blood sugar level risk of the user is. Therefore, the evaluation module 343 may evaluate the abnormal blood sugar level risk of the user by computing an average value of at least one difference between each of the user's at least one measured blood sugar level and the target blood sugar level range.

Further, when a meal period is used as the measurement time period, the blood sugar recording module 341 may obtain and record a before-meal blood sugar level and an after-meal blood sugar level of the user respectively, wherein the greater the difference between the before-meal blood sugar level and the after-meal blood sugar level is, the higher the abnormal blood sugar level risk of the user is, and vice versa, the smaller the difference between the before-meal blood sugar level and the after-meal blood sugar level is, the lower the abnormal blood sugar level risk of the user is. Therefore, the evaluation module 343 may respectively compute a plurality of differences between the before-meal blood sugar levels and the after-meal blood sugar levels measured on the same dates as the before-meal blood sugar levels, and then use an average value of the differences as a base for evaluating the at least one abnormal blood sugar level risk of the user within the at least one measurement time period. However, in other embodiments, the evaluation module 343 may apply other methods of evaluating abnormal blood sugar level risks to evaluate the user's abnormal blood sugar level risk within the measurement time period, and thus the embodiments of the present invention are not limited to the methods described above.

When the user sets up a plurality of measurement time periods, the processing element 340 may further include a sorting module 347 for sorting the abnormal blood sugar level risks of the user within the respective measurement time periods so as to generate a sorted risk list provided for display at the display element 350. Thus, through the sorted risk list shown at the display element 350, the user can understand within which measurement time periods he/she has higher abnormal blood sugar level risks. Subsequently, the user can manage his/her blood sugar levels with respect to the measurement time periods within which he/she has higher abnormal blood sugar level risks.

Further, the processing element 340 may further include a diet recording module 345 and a diet recommendation module 346. The diet recording module 345 may record diet records of the user made within the respective measurement time periods into the storage element 320, wherein the diet records may include a food list, a medicine list or other diet-related records showing what the user had eaten within the respective measurement time periods. The diet recommendation module 346 may select at least one date on which the at least one measured blood sugar level obtained for the at least one measurement time period falls within the target blood sugar level range as at least one target met date. Then, the diet recommendation module 346 may obtain from the storage element 320 the at least one diet record of the user made within the at least one measurement time period of the at least one target met date, thereby generating at least one recommended diet list in accordance with the at least one diet record obtained, wherein the recommended diet list is provided for display at the display element 350. In other words, the diet records of the user meeting the target blood sugar level range can be used as a diet reference for the user to follow in the future. Besides, since the recommended diet list displayed at the display element 350 is generated in accordance with the user's diet records, it can be better fit to the user's diet favorites or physical conditions.

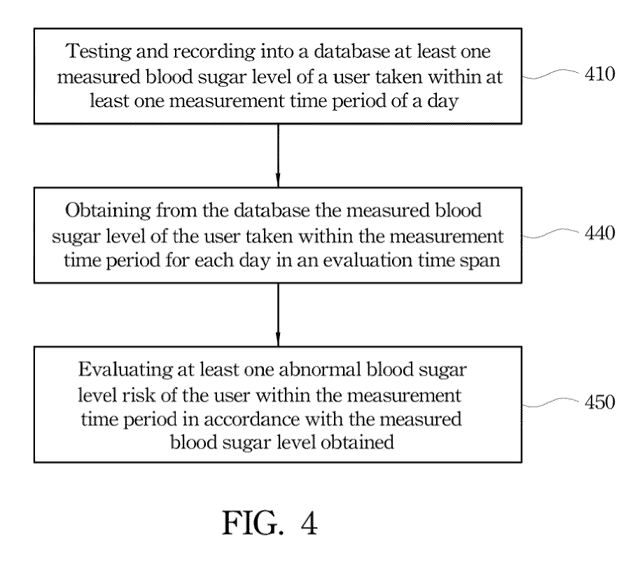

Referring to FIG. 4, FIG. 4 is a flow chart showing a blood sugar level analysis method according to another embodiment of the present invention. In a blood sugar level analysis method 400, at least one measured blood sugar level of a user is tested and recorded within at least one specific measurement time period, and the measured blood sugar level(s) of the user taken within the specific measurement time period(s) for each day in an evaluation time span is (are) used to evaluate at least one abnormal sugar level risk of the user within the specific measurement time period(s). The blood sugar level analysis method 400 can be enabled by using the aforementioned blood sugar level analysis system or device, or can be enabled as a computer program stored in a computer readable recording medium, so that the blood sugar level analysis method 400 can be performed after a computer reads the recording medium. The blood sugar level analysis method 400 includes the following steps.

In step 410, at least one measured blood sugar level of a user taken within at least one measurement time period of a day is tested and recorded into a database, wherein the setup of the measurement time period is just as described above.

In step 440, the measured blood sugar level of the user taken within the measurement time period for each day in an evaluation time span is obtained from the database. Further, the evaluation time span can be several ones of the dates listed in the database on which the measured blood sugar levels have been recorded. Besides, the evaluation time span can be set prior to step 440.

In step 450, at least one abnormal blood sugar level of the user within the measurement time period(s) is evaluated in accordance with the measured blood sugar level(s) obtained. The greater the difference between the user's measured blood sugar level and the target (normal) blood sugar level range is, the higher the abnormal blood sugar level risk of the user is, and vice versa, the smaller the difference between the user's measured blood sugar level and the target blood sugar level range is, the lower the abnormal blood sugar level risk of the user is. Therefore, in step 450, a target blood sugar level range is provided, and an average value of at least one difference between each of the user's at least one measured blood sugar level and the target blood sugar level range is computed, thereby evaluating the abnormal blood sugar level risk(s) of the user within the measurement time period(s).

Further, when a meal period is used as the measurement time period, step 410 may obtain and record a before-meal blood sugar level and an after-meal blood sugar level of the user respectively, wherein the greater the difference between the before-meal blood sugar level and the after-meal blood sugar level is, the higher the abnormal blood sugar level risk of the user is, and vice versa, the smaller the difference between the before-meal blood sugar level and the after-meal blood sugar level is, the lower the abnormal blood sugar level risk of the user is. Therefore, step 450 may respectively compute a plurality of differences between the before-meal blood sugar levels and the after-meal blood sugar levels measured on the same dates as the before-meal blood sugar levels, and then use an average value of the differences as a base for evaluating the at least one abnormal blood sugar level risk of the user within the at least one measurement time period. However, in other embodiments, step 450 may apply other methods of evaluating abnormal blood sugar level risks to evaluate the user's abnormal blood sugar level risk within the measurement time period, and thus the embodiments of the present invention are not limited to the methods described above.

Further, the blood sugar level analysis method 400 may include other steps. For example, when the user sets up a plurality of measurement time periods, the blood sugar level analysis method 400 may include a sorting step for sorting the abnormal blood sugar level risks of the user within the respective measurement time periods so as to generate a sorted risk list. Thus, through the sorted risk list, the user can understand within which measurement time periods he/she has higher abnormal blood sugar level risks. Subsequently, the user can manage his/her blood sugar levels with respect to the measurement time periods within which he/she has higher abnormal blood sugar level risks.

Further, the blood sugar level analysis method 400 may base on the user's historical diet records to recommend a diet list with better blood sugar level control to the user. Therefore, step 410 may additionally record the diet records of the user made within the measurement time periods to the database, wherein the diet records include a food list, a medicine list or other diet-related records showing what the user had eaten within the respective measurement time periods. Then, after step 450, at least one date on which the at least one measured blood sugar level obtained for the at least one measurement time period falls within the target blood sugar level range can be selected as at least one target met date. Thereafter, the at least one diet record of the user according to the at least one measurement time period of the at least one target met date is obtained from the database, thereby generating at least one recommended diet list in accordance with the at least one diet record obtained. In other words, the diet records of the user meeting the target blood sugar level range can be used as a diet reference for the user to follow in the future. Besides, since the recommended diet list is generated in accordance with the user's diet records, it can be better fit to the user's diet favorites or physical conditions.

It can be known from the embodiments of the present invention that the applications thereof can advantageously allow a user to learn his/her abnormal blood sugar level risks within the respective measurement time periods; further remind the user of the specific measurement time periods within which his/her blood sugar control is poor and needs further attention; and provide a recommended diet list in accordance with the user's diet record and measured blood sugar levels, thereby satisfying the user's personal diet favorites or physical conditions.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A blood sugar level analysis system, comprising:
a database;
an input device;
a processing device connected to the database and the input device, the processing device comprising:
a blood sugar recording module for obtaining at least one measured blood sugar level of a user taken within at least one measurement time period of a day, and recording the at least one measured blood sugar level into the database; and
an evaluation module for obtaining the at least one measured blood sugar level taken within the at least one measurement time period for each day in an evaluation time span from the database, and evaluating at least one abnormal blood sugar level risk of the user within the at least one measurement time period respectively in accordance with the at least one measured blood sugar level obtained; and
a display device connected to the processing device for displaying the at least one abnormal blood sugar level risk of the user within the at least one measurement time period.

2. The blood sugar level analysis system as claimed in claim 1, wherein the processing device further comprises:
a time span setting module for obtaining the evaluation time span from the input device.

3. The blood sugar level analysis system as claimed in claim 1, wherein the processing device further comprises:
a range setting module for obtaining a target blood sugar level range from the input device, wherein the evaluation module is used to obtain the at least one abnormal blood sugar level risk of the user within the at least one measurement time period by computing an average value of at least one difference between the target blood sugar level range and each of the at least one measured blood sugar level obtained.

4. The blood sugar level analysis system as claimed in claim 3, wherein the processing device further comprises:
a diet recording module for recording at least one diet record of the user made within the at least one measurement time period into the database; and
a diet recommendation module for selecting at least one date on which the at least one measured blood sugar level obtained for the at least one measurement time period falls within the target blood sugar level range as at least one target met date, and obtaining from the database the at least one diet record of the user made within the at least one measurement time period of the at least one target met date, and generating at least one recommended diet list in accordance with the at least one diet record obtained.

5. The blood sugar level analysis system as claimed in claim 1, wherein the processing device further comprises:
a sorting module for sorting the abnormal blood sugar level risks of the user within the measurement time periods when the number of the at least one measurement time period is greater than one, thereby generating a sorted risk list.

6. The blood sugar level analysis system as claimed in claim 1, further comprising:
a blood sugar testing device connected to the processing device for measuring the measured blood sugar level, and wherein the blood sugar recording module obtains the at least one measured blood sugar level of the user taken within the at least one measurement time period via the blood sugar testing device.

7. The blood sugar level analysis system as claimed in claim 1, wherein the blood sugar recording module obtains the at least one measured blood sugar level of the user taken within the at least one measurement time period via the input device.

8. A blood sugar level analysis method, comprising:
testing and recording into a database at least one measured blood sugar level of a user taken within at least one measurement time period of a day;
obtaining from the database the at least one measured blood sugar level taken within the at least one measurement time period for each day in an evaluation time span; and
evaluating at least one abnormal blood sugar level risk of the user within the at least one measurement time period respectively in accordance with the at least one measured blood sugar level obtained.

9. The blood sugar level analysis method as claimed in claim 8, wherein the step of evaluating the at least one abnormal blood sugar level risk of the user within the at least one measurement time period comprises:
providing a target blood sugar level range; and
using an average value of at least one difference between the target blood sugar level range and each of the at least one measured blood sugar level obtained as a base for evaluating the at least one abnormal blood sugar level risk of the user within the at least one measurement time period.

10. The blood sugar level analysis method as claimed in claim 8, further comprising:
providing a target blood sugar level range;
recording at least one diet record of the user made within the at least one measurement time period into the database; and
selecting at least one date on which the at least one measured blood sugar level obtained for the at least one measurement time period falls within the target blood sugar level range as at least one target met date;
obtaining the at least one diet record of the user from the database according to the at least one measurement time period of the at least one target met date; and
generating at least one recommended diet list in accordance with the at least one diet record obtained.

11. The blood sugar level analysis method as claimed in claim 10, wherein each of the at least one diet record of the user made within the at least one measurement time period comprises a food list or a medicine list.

12. The blood sugar level analysis method as claimed in claim 8, wherein the at least one measurement time period is at least one meal period, and each of the at least one measured blood sugar level obtained within at least one measurement time period comprises a before-meal blood sugar level taken before the meal period, and an after-meal blood sugar level taken after the meal period.

13. The blood sugar level analysis method as claimed in claim 12, wherein, when the number of the at least one measured blood sugar level obtained is greater than one, the measured blood sugar levels obtained comprises the before-meal blood sugar levels taken before each of the at least one meal period for each day in the evaluation time span, and the after-meal blood sugar level taken after each of the at least one meal period for each day in the evaluation time span; and wherein the step of evaluating the at least one abnormal blood sugar level risk of the user within the at least one meal period comprising:
respectively computing a plurality of differences between the before-meal blood sugar levels and the after-meal blood sugar levels measured on the same dates as the before-meal blood sugar levels; and
using an average value of the differences as a base for evaluating the at least one abnormal blood sugar level risk of the user within the at least one measurement time period.

14. The blood sugar level analysis method as claimed in claim 8, further comprising:
sorting the abnormal blood sugar level risks of the user within the measurement time periods when the number of the at least one measurement time period is greater than one, thereby generating a sorted risk list.

15. A computer-readable recording medium storing a computer program performing a blood sugar level analysis method, the blood sugar level analysis method comprising:
testing and recording into a database at least one measured blood sugar level of a user taken within at least one measurement time period of a day;
obtaining from the database the at least one measured blood sugar level taken within the at least one measurement time period for each day in an evaluation time span; and
evaluating at least one abnormal blood sugar level risk of the user within the at least one measurement time period respectively in accordance with the at least one measured blood sugar level obtained.

16. The computer-readable recording medium as claimed in claim 15, wherein the step of evaluating the at least one abnormal blood sugar level risk of the user within the at least one measurement time period comprises:
providing a target blood sugar level range; and
using an average value of at least one difference between the target blood sugar level range and each of the at least one measured blood sugar level obtained as a base for evaluating the at least one abnormal blood sugar level risk of the user within the at least one measurement time period.

17. The computer-readable recording medium as claimed in claim 15, further comprising:
providing a target blood sugar level range;
recording at least one diet record of the user made within the at least one measurement time period into the database; and
selecting at least one date on which the at least one measured blood sugar level obtained for the at least one measurement time period falls within the target blood sugar level range as at least one target met date;
obtaining the at least one diet record of the user from the database according to the at least one measurement time period of the at least one target met date; and
generating at least one recommended diet list in accordance with the at least one diet record obtained.

18. The computer-readable recording medium as claimed in claim 15, wherein the at least one measurement time period is at least one meal period, and each of the at least one measured blood sugar level obtained within at least one measurement time period comprises a before-meal blood sugar level taken before the meal period, and an after-meal blood sugar level taken after the meal period.

19. The computer-readable recording medium as claimed in claim 18, wherein the number of the at least one measured blood sugar level obtained is greater than one, and the measured blood sugar levels obtained comprises the before-meal blood sugar levels taken before the meal period for each day in the evaluation time span, and the after-meal blood sugar level taken take after the meal period for each day in the evaluation time span; and wherein the step of evaluating the at least one abnormal blood sugar level risk of the user within the at least one meal period comprising:
respectively computing a plurality of differences between the before-meal blood sugar levels and the after-meal blood sugar levels measured on the same dates as the before-meal blood sugar levels; and
using an average value of the differences as a base for evaluating the at least one abnormal blood sugar level risk of the user within the at least one measurement time period.

20. The computer-readable recording medium as claimed in claim 15, further comprising:
sorting the abnormal blood sugar level risks of the user within the measurement time periods when the number of the at least one measurement time period is greater than one, thereby generating a sorted risk list.
